(19) United States
(12) Patent Application Publication
(10) Pub. No.: US 2014/0343613 A1
(43) Pub. Date: Nov. 20, 2014

(54) BONE ANCHORING MEMBER WITH CLAMP MECHANISM

(71) Applicants: Kenneth Arden Eliasen, Wrentham, MA (US); Mark Ettlinger, Lexington, MA (US); Kamran Aflatoon, Corona del Mar, CA (US)

(72) Inventors: Kenneth Arden Eliasen, Wrentham, MA (US); Mark Ettlinger, Lexington, MA (US); Kamran Aflatoon, Corona del Mar, CA (US)

(21) Appl. No.: 13/897,003

(22) Filed: May 17, 2013

(51) Int. Cl. A61B 17/70

(52) U.S. Cl. CPC A61B 17/7034; A61B 17/705
USPC 606/278

(57) ABSTRACT

A multi-piece disc replacement implant for replacing a disc removed by a discectomy including an upper plate member, a lower plate member, and an intermediate resilient member providing movement between te two plate members replicating the natural movement of the spine. The plate members are rigid and have orthogonal sidewalls forming an enclosure. The resilient member is an elastic solid or a multi-chamber balloon structure of fluid-filled sacks that collectively define a non-uniform shape such as an oblate spheroid, or a helically coiled string of beads. Such an implant is capable of supporting the compressive and cyclic loads required of a natural disc. The upper and lower plate members are cooperatively formed to selectively limit the allowable range of motion in any given direction and a provided with protrusions to be received in one or more channels cooperatively formed in the vertebrae and secured in place by a bone screw.

### CROSS-REFERENCE TO RELATED APPLICATIONS

The present application is a continuation of co-pending U.S. patent application Ser. No. 12/653,086 for a “Bone Anchoring Member With Clamp Mechanism” filed Dec. 8, 2009 from which priority is derived and which is incorporated herein by reference. U.S. patent application Ser. No. 12/653,086 is itself a continuation-in-part of U.S. patent application Ser. No. 12/462,127 for “Bone Anchoring Member” filed Jul. 29, 2009 which is also incorporated herein by reference and which derives priority from U.S. provisional application 61/137,255 filed on Jul. 29, 2008 which is further incorporated herein by reference.

### BACKGROUND OF THE INVENTION

1. Field of the invention

The present invention relates generally to devices and methods for treating spinal disorders and more specifically to a fixation assembly for retaining vertebrate endoskeletal members in a desired fixed spatial relationship.

2. Description of the Background

A variety of devices are known for the fixation of endoskeletal members or bones in humans and animals. Fixation of bones may be temporary in order to allow for normal healing, as with a break in the long bones of the limbs, or permanent in order to provide support and alignment of the skeletal members. The latter is often the case with respect to the vertebrae of a spinal column where support and spatial fixation are necessary due to injury or disease. Even where vertebral or spinal healing occurs after fixation, the means and devices of fixation often remain in position for life. Such fixations means and devices generally include hardware such as bone fasteners, plates, rods and connectors.

Bone fasteners utilized in conjunction with spinal fixation often include a pedicle screw or screws that are anchored by threaded engagement into the pedicle of each of the vertebrae that are to be maintained in a desired spatial relationship along with one or more connector assembly or assemblies for engaging implanted support rods or plates with the screw. One or more longitudinal support rods generally extending longitudinally along the spinal column is connected securely to the pedicle screw by the assembly in a manner that allows the vertebrae to be secured and maintained in a desired alignment. In order to achieve the desired stability, the bone fasteners must be attached securely to the vertebrae and connected firmly to the rod and/or one another.

A variety of means have been utilized to connect the rod to the pedicle screws or other bone fasteners. The secure placement and alignment of a rod between pedicle screws or the placement of a plate linking two or more firmly implanted screws can be complicated by spinal geometry and the angular orientation of the screws and exacerbated by any deformity of the spinal column. Precise alignment with all of the bone fasteners and secure connections is desirable to decrease the possibility that unanticipated and undesired stresses will cause the bone bodies or vertebrae to fracture or the screws to loosen over time. It is, therefore important that bone fastener assemblies be provided and implanted so as to minimize the likelihood of the establishment of undesirable stresses.

Thus, it is an object of the present invention to securely place, retain and align a rod or plate with a pedicle screw or screws over the primary longitudinal axis of the pedicle screw in order to minimize the likelihood of the establishment of undesirable stresses and to provide for ease of installation.

It is further an object of the present invention to provide a bone anchor that is durable and easy to implant yet which provided the surgeon with the versatility to adapt to a wide variety of spinal conditions.

### SUMMARY OF THE INVENTION

Accordingly, there is provided a pedicle screw for insertion into the pedicle portion of a vertebra and having at its exposed end a threaded rod connected in a generally parallel orientation to the longitudinal axis of the pedicle screw but laterally offset from the longitudinal axis of the pedicle screw. The threaded rod may be secured directly to the screw but is preferable provided with a collar that is captured by an enlarged screw head to seat, orient and retain the threaded rod in position. A locking arm and retaining nut are provided over the threaded rod for affixing a spinal rod to the pedicle screw substantially over and aligned with the longitudinal axis of the screw. A yoke may be provided in the collar also to receive and secure the rod. An alternate cap and clamping elements enable the incorporation of cross links between multiple screws and/or rods.

The foregoing objects, features and attendant benefits of this invention will, in part, be pointed out with particularity and will become more readily appreciated as the same become better understood by reference to the following detailed description of a preferred embodiment and certain modifications thereof when taken in conjunction with the accompanying drawings.

### BRIEF DESCRIPTION OF THE DRAWINGS

In the drawings:

FIG. 1 is a perspective view of an embodiment according to the present invention.

FIG. 2 is a front elevation view of an embodiment according to the present invention after removal of the stem.

FIG. 3 is a front perspective view of an embodiment according to the present invention after removal of the stem.

FIG. 4 is a rear perspective view of an embodiment according to the present invention after removal of the stem.

FIG. 5 is an elevation view of an embodiment according to the present invention.

FIG. 6 is a section through the elevation of FIG. 5.

FIG. 7 is a partial detail view of the section of FIG. 6.

FIG. 8A is a perspective view of the cap.

FIG. 8B is side view of the cap.

FIG. 8 is a bottom view of the cap.

FIG. 9A is perspective view of the cup.

FIG. 9B is partial section view of the cap.

FIG. 10A is perspective view of the screw.

FIG. 10B is top view of the screw.

FIG. 10C is an elevation of the screw.

FIG. 11A is top view of the yoke.

FIG. 11B is perspective view of the yoke.

FIG. 11C is side view of the yoke.

FIG. 11D is a section view of the yoke at cut A-A of FIG. 11C.

FIG. 12 is a perspective view of an alternate embodiment according to the present invention.

FIG. 13 is a section view of an alternate embodiment according to the present invention.

FIG. 14 is a side view of an alternate embodiment according to the present invention.

FIG. 15 is a perspective view of an element of the alternate embodiment of FIG. 16.

FIG. 16 is a perspective view of an alternate embodiment according to the present invention.

FIG. 17 is a perspective view of an alternate embodiment according to the present invention.

FIG. 18 is a perspective view of an alternate embodiment according to the present invention.

FIG. 19 is a perspective view of the nut.

FIG. 20 is a transparent side view of the nut.

FIG. 21A is perspective view of an alternate embodiment of the present invention.

FIG. 21B is a top view of an alternate cap according to the present invention.

FIG. 21C is a side view of an alternate cup according to the present invention.

### DETAILED DESCRIPTION OF THE PREFERRED EMBODIMENTS

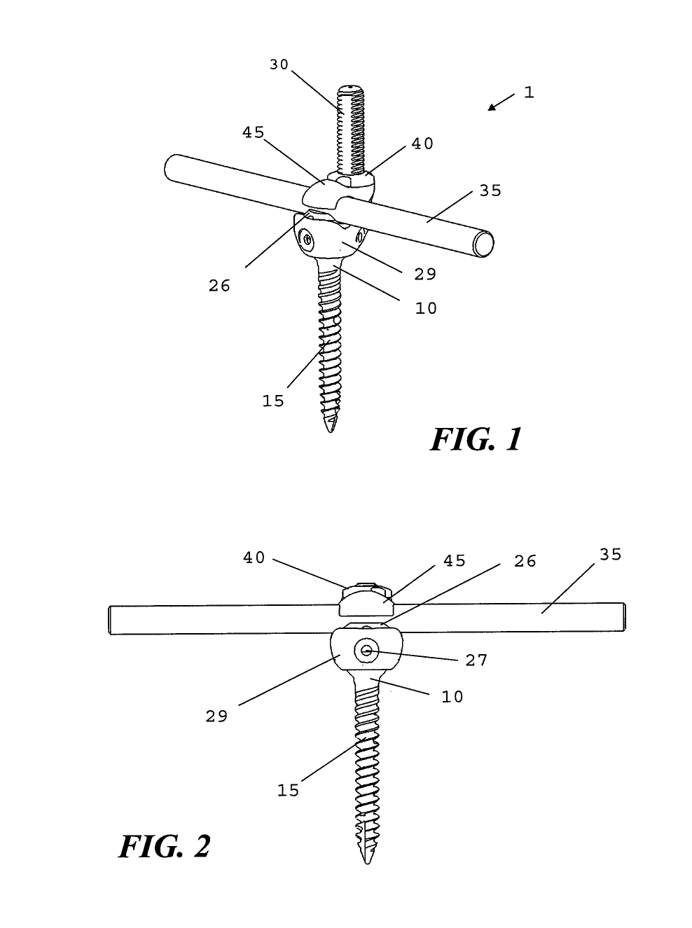

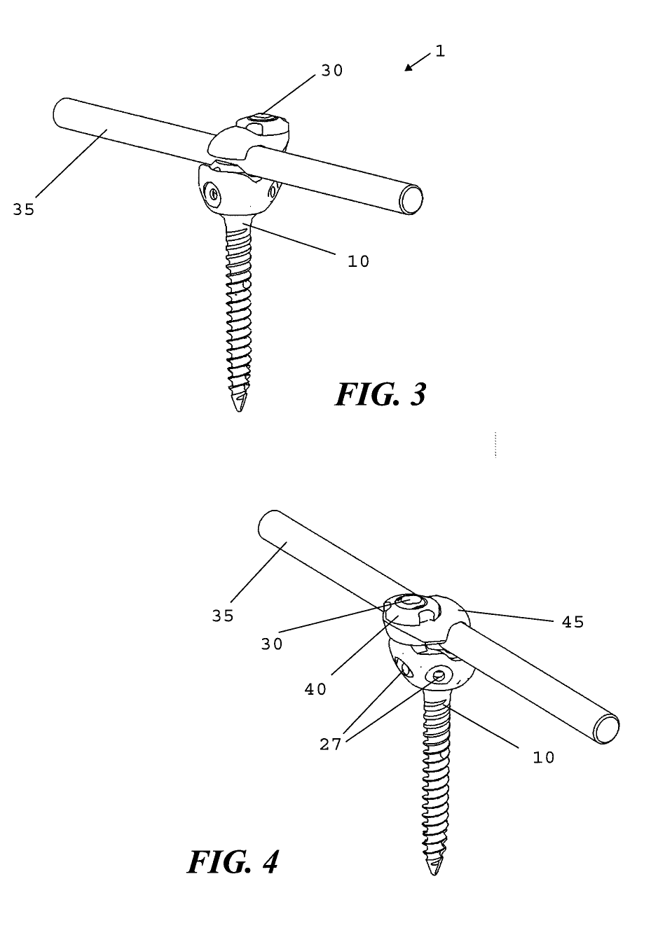

With reference to FIGS. 1-6, a preferred embodiment of the present invention 1 comprises a pedicle screw 10 for insertion into the pedicle portion of a vertebra and having at its exposed end a threaded rod or stem 30 connected in a generally parallel orientation to, but laterally offset from, the longitudinal axis of the pedicle screw 10. It is observed that for purposes of this application the longitudinal axis of the pedicle screw 10 is defined as the vertical axis and references to the vertical are made with respect to this axis. The figures provided herewith generally depict the pedicle screw on the vertical axis although it is, of course, understood that the axis of the pedicle screw is unlikely to be vertical one implanted in the vertebra of a patient. Thus, the stem 30 is substantially vertical when connected to the pedicle screw 10. The stem 30 is preferable provided as part of a cup assembly 29 that is captured by an enlarged head of screw 10 and may include a yoke 26 for receiving spinal rod 35. A cap 45 and nut 40 are provided over stem 30 securing the spinal rod 35 in the yoke 26 and compressing and securing the entire assembly. A distal portion of stem 30 may be remove after nut 40 has been secured in place as seen in FIG. 2.

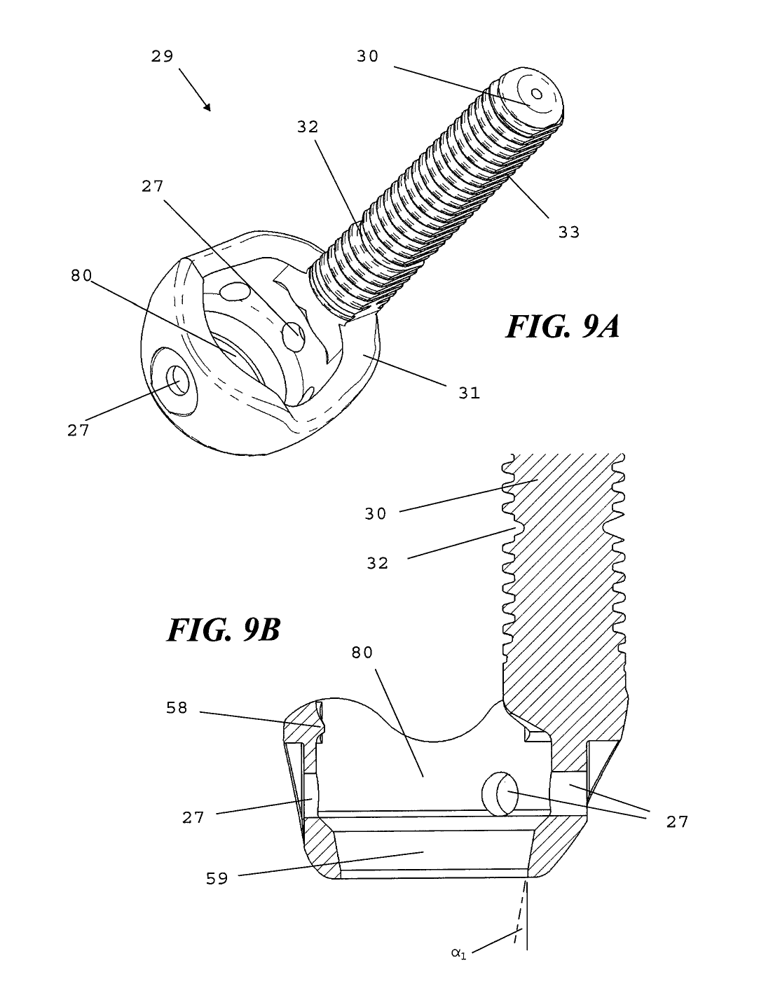

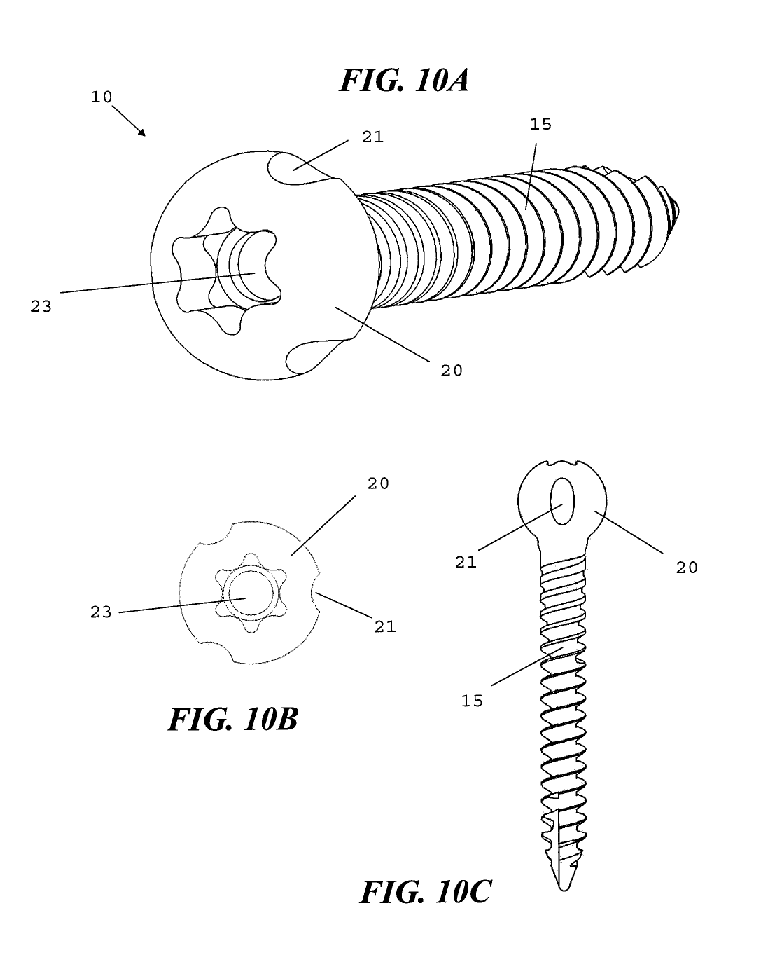

With specific reference to FIGS. 10A-10C, screw 10 is provided with a threaded shaft 15 for engagement with a bore hole prepared in the pedicle region of the vertebra. Threaded shaft 15 may be of any known bone screw design and is depicted in a preferred form which is tapered to a self tapping point for insertion into the prepared hole. A generally spherical head 20 is provided at the exposed end of shaft 15. Head 20 has a diameter greater than the major diameter of the threaded shaft 15 as seen in FIG. 10C and is preferably round in plan view (FIG. 10B) but may be provided with a series of side cut features 21 for positive engagement with other elements of the device further described below. Side cut features 21 are preferably evenly spaced radially about the longitudinal axis of the shaft 15 as extended through the head 20. Three such side cut features 21 are depicted in a preferred embodiment as seen in FIG. 10B but a greater or smaller number may be employed. As depicted in FIG. 10A, a keyed (non-round) recess 23 is provided in the top surface of the head 20 coaxial with the longitudinal axis of the threaded shaft 15 in order to facilitate rotational insertion of the screw 10 into the bone by insertion of a cooperative tool such as a hexalobe or Allen wrench into the recess.

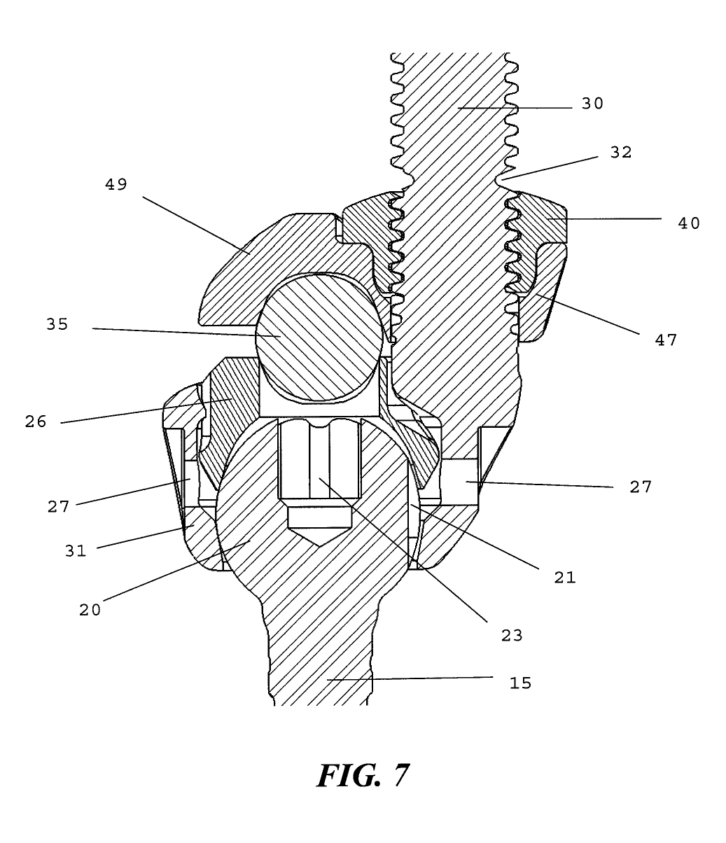

With reference to FIGS. 9A and 9B, a cup 29 having a threaded stem 30 affixed to a collar 31 is provided. Prior to rotational insertion of the screw 10 into the bone as described above the shaft 15 of the screw 10 is inserted through the generally circular central bore 80 of the collar 31 from above, as seen in FIG. 6. The screw 10 is advanced through the central bore until the screw head 20 enters the central bore. The diameter of the screw head 20 is small enough to enter the central bore 80 from above but is too large to pass through the tapered lower exit opening, as seen in FIG. 7. The lower exit opening is provided with an annular tapered lip 59 having a maximum diameter large enough to receive the head 20 but tapered to a minimum diameter smaller than the diameter of the head 20 such that the cup is captured on the threaded shaft 15 with the cup contacting the screw head only at the annular tapered lip, as seen in FIG. 6 as well as in FIG. 7. So captured, the longitudinal axis of the stem (the stem axis) is vertical although it may deviate from the vertical by up to 30degrees at the discretion of the surgeon. The taper angle αₗ of lip 59 is preferably from 18° to 22° from the stem axis where it to engages the screw head 20 to induce deformation at the side cut features 21 under applied compressive force of the nut 40 on stem 30, as further described below.

A series of counter bores 27 radially oriented through the walls of the collar 31 into the central bore 80 are preferably provided to engage a rocker arm or reducer tool as an aid in seating the spinal rod 35 during implantation. Counter bores 20 may also act as points of engagement for additional elements of one or more alternate embodiments of the present invention as further described below. Stem 30 is provided extending upward from the collar 31 of cup 29 so as to be parallel to the central axis though the bore 80 and is threaded on its external surface, the threading preferably being #12-28 (¼-20) ACME having rounded cornices. Stem 30 is provided with a break off feature partially along its length to facilitate removal of the distal end of the stem after implantation. The break-off feature is preferably a circumferential score 32 at the desired point of removal although any known means of facilitating break-off may be employed. Stem 30 is also preferably provided with opposing flat sides 33 to facilitate positive orientation of the cap 45 as described below.

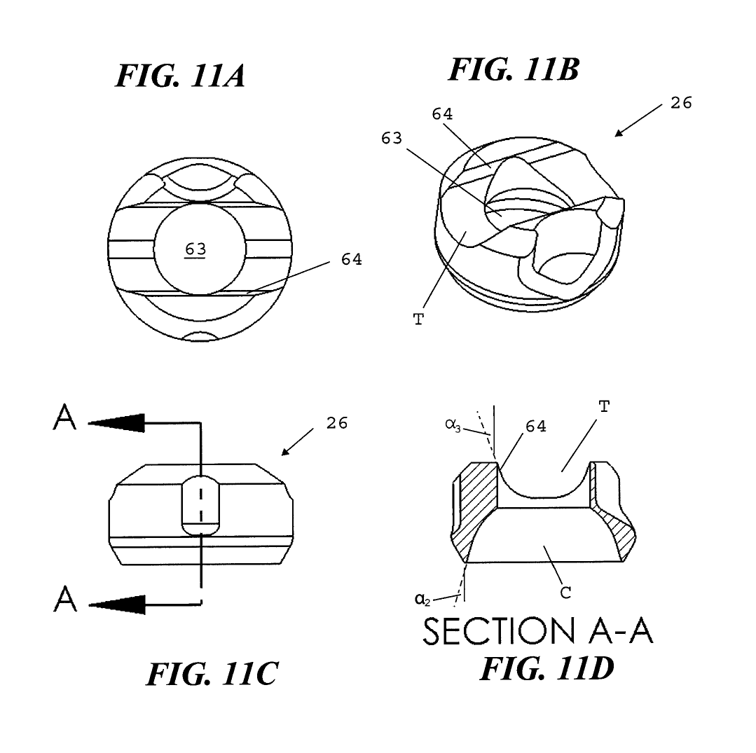

With reference to FIGS. 11A-11D, after the screw and cup subassembly are joined a yoke 26 is provided within the central bore 80 of the collar for seating and engaging the spinal rod 35. Yoke 26 may be inserted after the screw/cup subassembly is implanted but is preferably preassembled, a hole 63 being provided to permit insertion of a tool into keyed recess 23 of head 20 to facilitate rotational insertion of the shaft 15 into the bone. A protrusion or lip 58 (see FIG. 9B) inside the central bore 80 of the cup 29 is provided to retain the yoke after preassembly. Yoke 26 is provided with an upper surface contoured for engagement with the spinal rod 35 and a lower surface contoured for engagement with the head 20 of screw 10. The upper surface contour T (FIG. 11D) is generally concave on an axis perpendicular to the stem to receive and engage a spinal rod 35 having a circular cross section. However the shape of the concave surface may be altered to accommodate an alternate rod cross section under the principals disclosed here. More specifically, circular rod 35 engages yoke 26 at the tapered edges 64 of the semi-circular trough T, as seen in FIG. 7. The upper edges 64 are provided with a taper having an angle, α3, from 36° to 40° (FIG. 11D) from the stem axis, again to facilitate deformation of the assembly under compression of nut 40 which enhances the strength of the completed assembly. Yoke 26 may be keyed for positive orientation within the central bore 80 and ensure alignment of the trough T with the desired axis of spinal rod 35. Similarly, the bottom surface of the yoke 26 is provided with a cup shaped recess C (FIG. 11D) in which to receive the rounded top of generally spherical head 20. The rim of the cup recess C is provided with a conical taper preferably having an angle, α₂, from 36° to 40° from the stem axis to engage the upper surface of head 20 (FIG. 7) under the described applied compressive force of the nut 40. The tapered and other contact surfaces described may be provided with a surface texture such as knurling to enhance engagement.

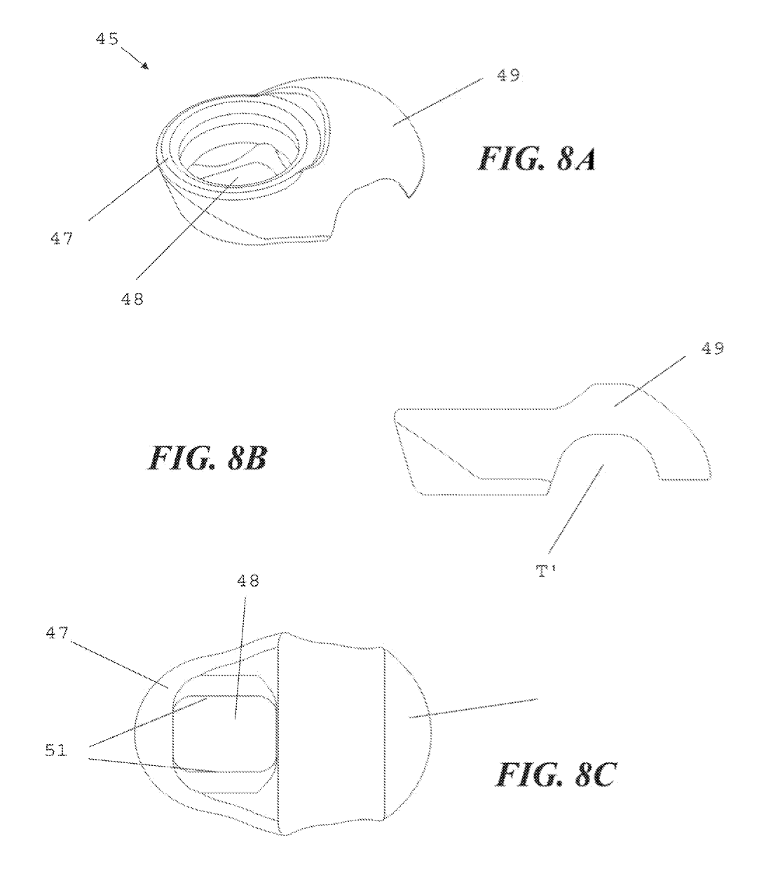

With reference to FIGS. 8A-8C, once the screw/cup subassembly is implanted with the yoke 26 in position, the spinal rod 35 is positioned within the trough T. As noted, and if necessary, a rocker arm, reducer tool or similar may engage the lateral bores 27 to position the rod 35 in the trough T. Once positioned the rod 35 is retained by a cap 45 having a collar 47 and a rod arm 49. The rod arm 49 is preferably formed at its lower surface with a concave trough shaped recess T′ (FIG. 8B), complimentary to trough T in the yoke 26, in which to receive and engage the top surface spinal rod 35. The rim of the trough T′ of the rod arm 49 may also be provided with a linear taper preferably from 36° to 40° from the stem axis to engage the upper surface of rod 35 or may be provided with a profile that generally matches the upper surface of the cross section of the rod 35.

The collar 47 of the cap 45 is generally circular with central aperture 48 for receiving the distal end of stem 30. Central aperture 48 is sized to receive stem 30 with little play and is provided with opposing flat sides 51 for cooperative engagement with the flat features 33 of the stem 30. The trough T′ of rod arm 49 is generally aligned with trough T of yoke 26 and the longitudinal axis of the spinal rod 35 when the flat features 33 and 51 of stem 30 and aperture 48, respectively, are cooperatively engaged. The upper surface of the collar 47 of the cap 45 is preferably formed with a recess in which to receive at least a portion of nut 40 so as to reduce the overall projection of the device from the bone surface.

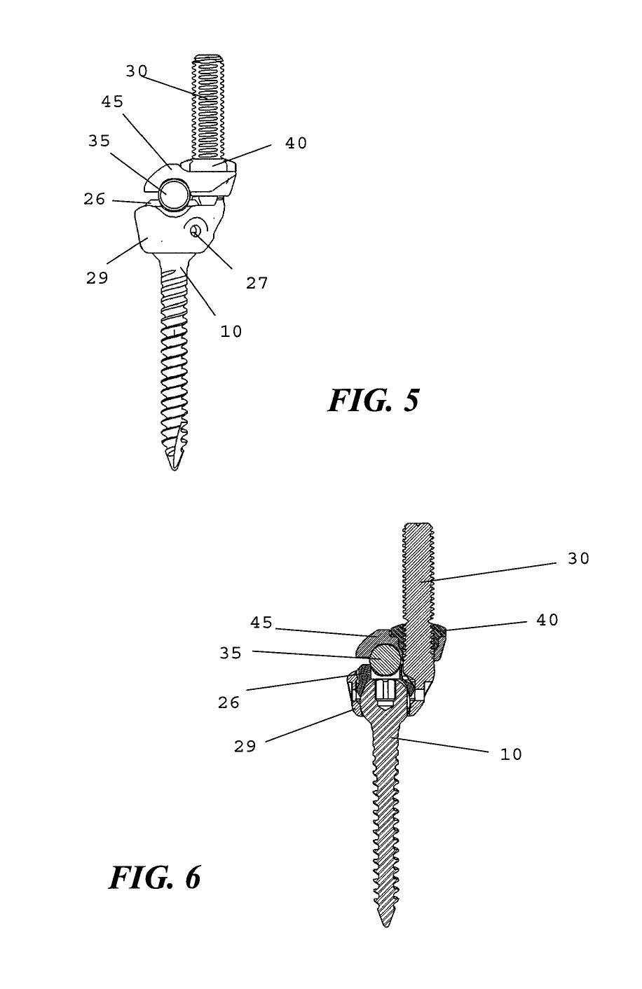

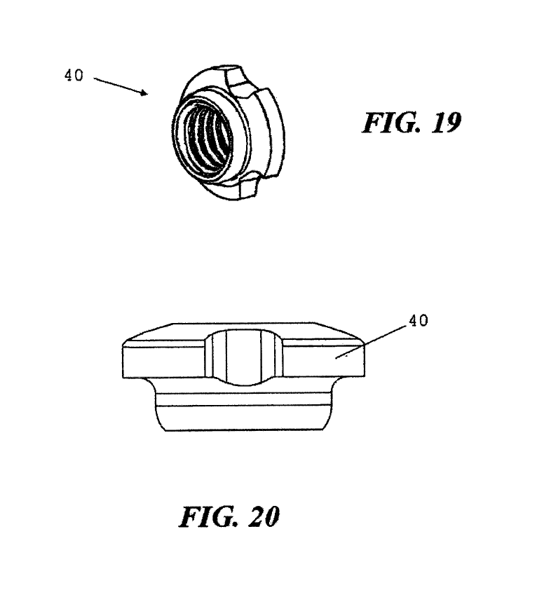

With reference to FIGS. 5, 6, 19 and 20, a flange type nut 40 is threaded on to stem 30 and advanced to engage cap 45 and secure the rod 35 between the yoke 26 and the cap 45. Nut 45 is preferably provided with non-standard #12-28 (¼-20) ACME internal threading having rounded cornices or fillet radiuses on the internal and external thread form edges for engaging threaded stem 30, although any threading formed for complimentary engagement with the threading of the stem will suffice albeit with reduced assembly strength.

As described, prior to implantation the screw 10, cup 29 and yoke 26 are preferably preassembled. After surgically reaching the implantation site a borehole is prepared into the pedicle region of the subject vertebra and the shaft 15 inserted. A tool such as a hexalobe driver inserted into the drive recess 23 in the screw head 20 via the yoke hole 63 to rotationally advance the shaft into the borehole until the lower surface of the collar 31 of cup 29 approaches the surface of the bone but is not constrained by contact with the surface. Stem 30 is generally parallel to the vertical axis and/or normal to the bone surface although the orientation of the cup 29 and stem 30 may be adjusted at this point as necessary to achieve the desired alignment. The spinal rod 35 is positioned in line with the trough of the upper side of the yoke 26 and a cap 45 is slid onto stem 30 such that the flat alignment surfaces 33 and 51 orient the trough of the rod arm 49 over the rod 35. Nut 40 is threaded onto stem 30 and rotated to draw the cap 45 down the stem and bring the rod 35 with it until rod 35 contacts the trough of yoke 26. The extended stem 30 permits the surgeon to use the action of nut 40 to draw the rod 35 into the yoke 26. In certain situations it may be necessary to use additional tools to gain leverage on the rod 35 in order to seat it in the yoke. Counter bores 27 in collar 31 provide a point of engagement for such tools

Once the rod 35 has been brought to the yoke 26 by cap 45 and the cup adjusted to the surgeon's satisfaction, nut 45 is further rotated to compress the assembly. The compressive force of the nut 45 generates hoop stresses in the conical taper of the annular lip 59 in the collar 31 of the cup 29 about the screw head 20 causing the stresses at the side cut features 21 of head 20 to exceed the material yield strength and the annular lip to deform. The result is a passive lock in the form of an indent (screw head) and detent (cup). Similar hoop stresses are generated in the conical taper C of the underside of yoke 26 resulting in a similar passive lock in the form of an indent (screw head) and detent (yoke). As a result the pedicle screw 10, cup 29 and yoke 26 are mechanically secured together as a single unit. The taper of the trough of the upper surface of the yoke 26 (and possibly cap 29) deforms in a similar manner on the rod 35 to retain the rod in position although deformation occurs in to a considerably lesser degree between these components. Once nut 45 is tightened to fully compress and seat the components as described the distal portion of stem 30 beyond the break off feature 32 may be snapped off such that the new distal end stem 30 is generally flush with the upper surface of nut 40.

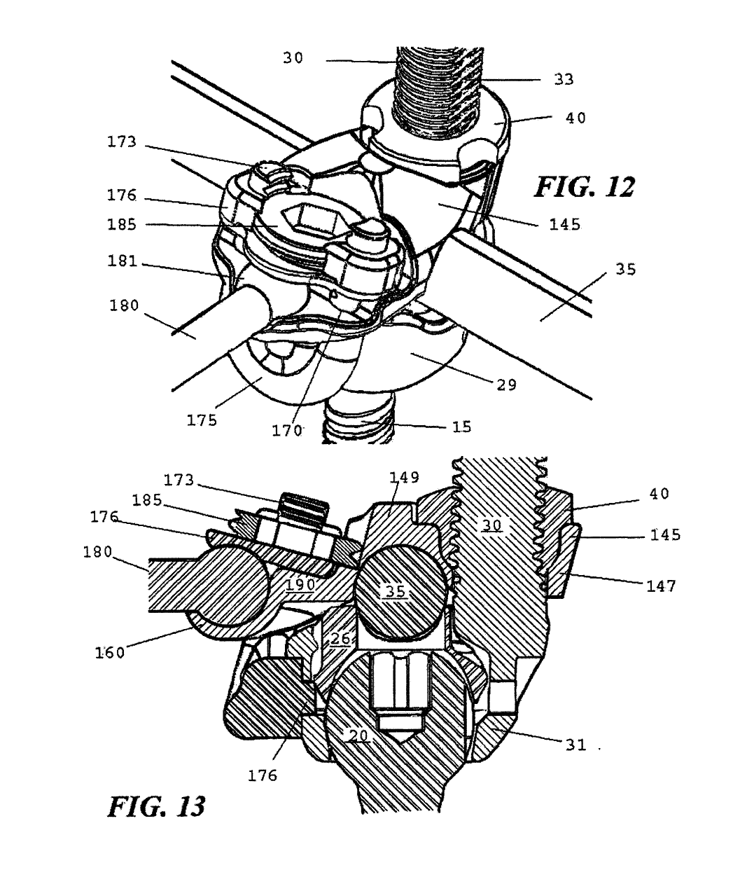

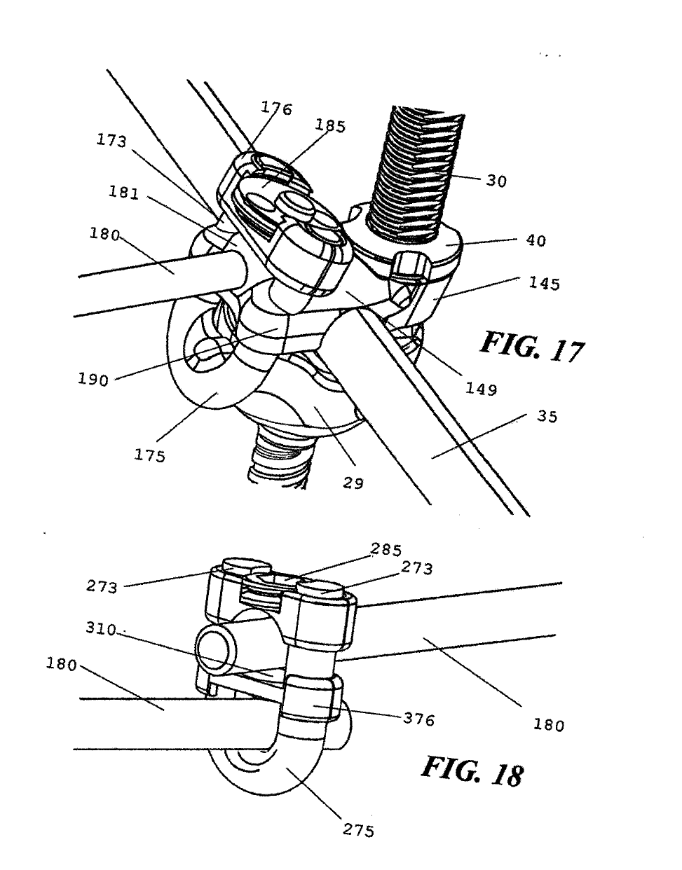

It may be desirable in some cases for a surgeon to cross-link a pair of pedicle screw assemblies according to the present invention implanted in left and right pedicles. An alternate embodiment of the present invention is depicted in FIGS. 12-18 to provide for such a situation, the alternate embodiment being substantially similar to recited preferred embodiment with the exception of an alternate to cap 45 and additional elements to facilitate attachment of a cross-link connector 180. Alternate cap 145 is provided to replace cap 45 and is similarly provided with a collar 147 and a rod arm 149. Collar 147 is formed as a circular member with central aperture 148 (not shown) for receiving the distal end of stem 30. Central aperture 148 is sized to receive stem 30 and is provided with opposing flat sides 151 (not shown) for cooperative engagement with the flat features 33 of stem 30 such that the rod arm 149 is automatically oriented with respect to the upper surface of yoke 26 to receive and retain the rod 35. The upper surface of the collar portion 147 of the cap 145 is again preferably formed with a recess to receive at least a portion of nut 40 so as to reduce the overall projection of the device from the bone surface. Rod arm 149 is preferably formed at its lower surface with a trough shaped recess in which to receive and engage the top surface spinal rod 35. The rim of the trough of rod arm 149 may also provided with a linear taper preferably from 36° to 40° as measured from the stem angle.

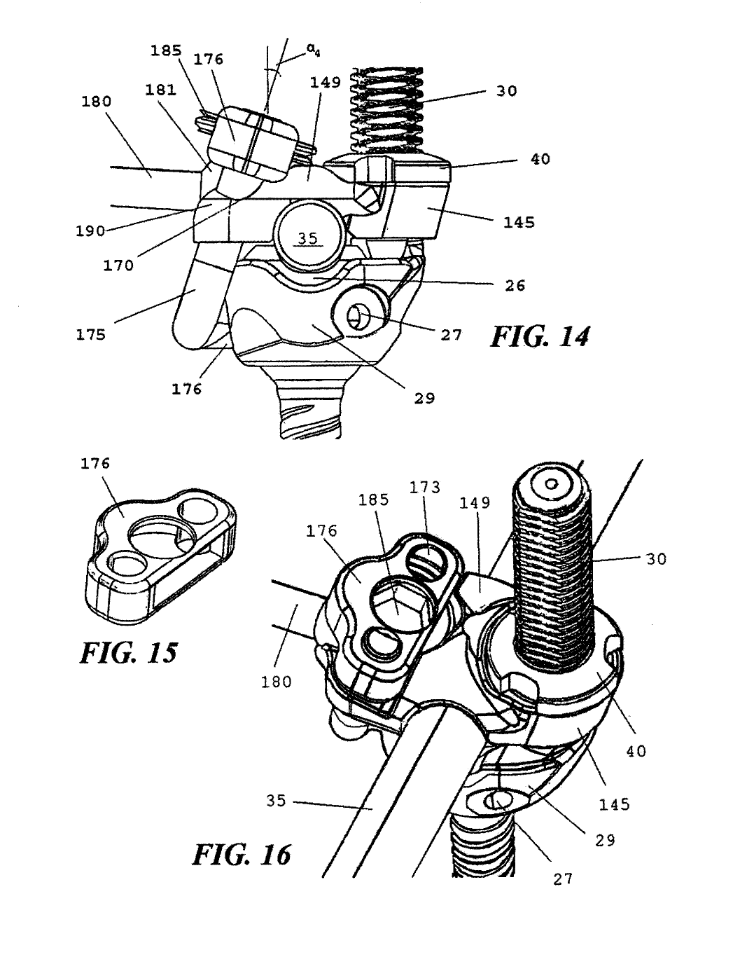

The distal portion 190 of the alternate rod arm 149 is elongated as compared to the previously described rod arm 49 and extends the past spinal rod 35, as seen in FIG. 14. The top surface of the distal portion 190 is formed with a ball socket 160 having a semi-spherical bottom for receiving a ball end 181 of cross-link 180, as seen in FIG. 13. Ball socket 160 is preferably formed along a centerline of cap 145 (as viewed from above) in substantial alignment with the longitudinal axes of stem 33 and shaft 15 (see FIG. 12). A lateral channel is provided into the ball socket 160 to permit the cross-link 180 to enter. The lateral channel widens as it leaves the ball socket 160 to permit articulation of the cross-link 180 up to 25° in any direction from the centerline.

The ball socket 160 is flanked on either side by through-holes 170. A U-clamp 175 is provided such that one distal arm 173 of the U extends through each through-hole 170. The inner surface of each arm 173 is cooperatively threaded to receive an externally threaded nut 185 between them. Externally threaded nut 185 is alternately described as a set screw, blind screw, or grub screw. A locking plate 176 is provided over the distal arms 173 and beneath nut 185, the lower surface of which is formed to engage the ball 181 of the cross-link 180 and secure it in the socket 160 under force of the nut 185. The U-clamp 175 is further provided with a pin 176 on one side of its proximal end at the base of the U, as seen in FIG. 13. The pin 176 engages lateral bore 27 of the cup 29 (see FIG. 9a) to secure the U-clamp to the assembly. As seen in FIG. 14, the arms 173 of the U-clamp are angled back towards the stem 30 so as to maintain pin 176 in lateral bore 27 as nut 185 is tightened. The U-clamp angle, α₄, is preferably between 20° and 35° (see FIG. 14). The through holes 170 are cooperatively angled. As seen in FIGS. 15 and 16, locking plate 176 may alternately be formed to enclose nut 185 and to permit distal arms 173 to pass through as the nut is advanced.

As stated, the described alternate embodiment is utilized when it is desirable in the view of the surgeon to cross-link a pair of pedicle screw assemblies according to the present invention implanted on either side of the spine. The cross-link is preferably one or more rods 180 or cables having a ball end 181. Where multiple rods are utilized to join to pedicle screw assemblies due to spinal anatomy or deformity the rods must be securely and rigidly joined. FIG. 18 depicts an additional element of the present invention for joining cross-link rods 180 utilizing a U-clamp 275 substantially similar to U-clamp 175.

As above, U-clamp 275 is formed such that the inner surface of each distal arm 273 is cooperatively threaded to receive an externally threaded nut 285 between them. An upper locking plate 276 is provided on its upper side with a central void 260 for rotatably retaining nut 285. Void 260 flanked by through holes 270 for slideably receiving distal arms 273, the through holes intersecting the central void 260 such that the threaded surfaces of arms 273 are engaged by nut 285. The lower surface of the upper locking plate 276 is contour to cooperatively engage the surface of a cross-link 180. A medial locking plate 376 is similarly provided with through holes 370 for slideably receiving the distal arms 273. The upper and lower surfaces of the locking plate 376 are provided with a detent 310 between through holes 370 for engaging the surface of a first and second cross link above and below the medial locking plate 370, respectively. Detents 310 is preferably a linear protrusion having a tapered or triangular cross section. When assembled as depicted in FIG. 18 with the cross links 180 positioned as desired by the surgeon, nut 285 is rotated to advance the upper locking plate 276 down the distal arms 273. The upper and lower cross links 180 are thus compressed between the upper locking plate 276 and the lower curve of the U-clamp causing the detents 310 of the intermediate locking plate 376 to engage and deform the surface of the cross links creating an indent, mechanically securing them together as a single unit. The result is a passive lock in the form of an indent (cross link) and detent (intermediate locking plate) that securely joins the cross links and by extension the left and right pedicel screws of the present invention.

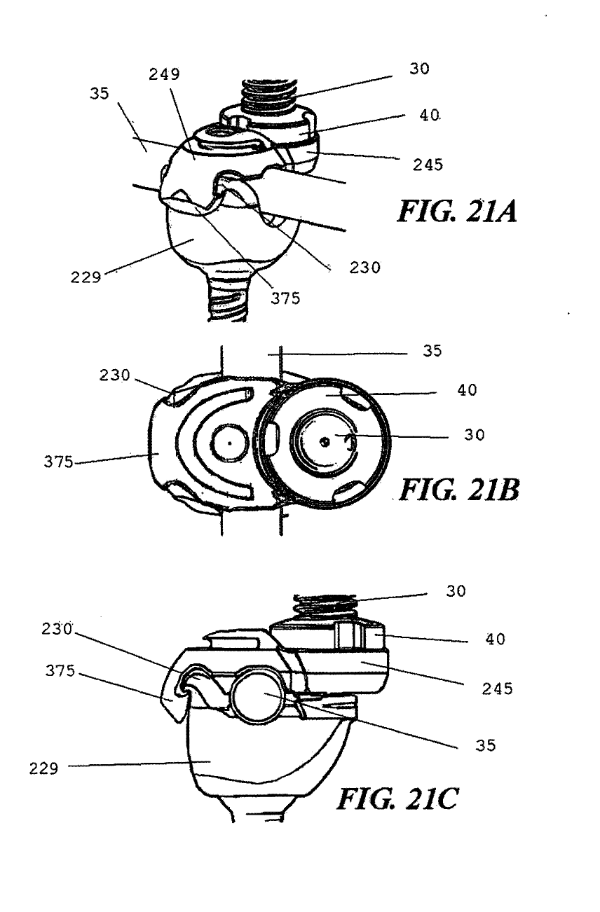

With reference to FIGS. 21A-21C, yet another alternate embodiment is provided. The embodiment of FIGS. 21 provides for additional stability of the cap in the absence of a cross through a cap 245 and cup 129 of alternate design. All other elements are substantially similar to those described with respect to the embodiment of FIG. 1. Cap 245 is provided with a hooked end 375 extending from the distal portion of rod arm 249. Hooked end 375 descend to and engages an integrally formed lip 230 on the collar of the cup 229. The engagement of the hooked end 375 and lip 230 may be along a straight line substantially parallel to the rod 35 in order to minimize the stresses internal stresses or may be in the form of an arc about a vertical axis which provides greater ease of installation but higher localized stress points. The addition of hooked end 375 and lip 230 provides additional stability to cap 245 allowing a reduction and smoothing of the profile of rod arm 249.

It should be understood that the disclosure may be used with a variety of pedicle screw designs and sizes. It should also be understood that the disclosure may be constructed of a variety of suitable surgical grade materials including stainless steel and titanium as well as composite materials having suitable strength and corrosion resistance properties should such materials be approved for surgical implantation. This application is therefore intended to cover any variations, uses, or adaptations of the invention using its general principles. Further, this application is intended to cover such departures from the present disclosure as come within known or customary practice in the art to which this invention pertains.

I claim:

1. A bone anchoring member with cross link connector comprising:
a bone anchor comprising
a first shaft having a threaded first end for insertion into a vertebra and a second end;
a head affixed to said second end;
a second shaft affixed to said head substantially parallel to but laterally offset from the longitudinal axis of said first shaft;
a cap having a retaining arm and a collar, said collar adapted for receiving said second shaft, said cap further comprising a semispherical recess for receiving a ball end of a cross-link, and at least one though-hole extending from a lower surface of said cap to an upper surface of said cap;
a first nut for threaded engagement with said second shaft whereby said cap may be advanced down said second shaft to retain a rod positioned between said head and said retaining arm;
a clamping member engaging said head and having at least one threaded arm extending though said through-hole of said cap,
a locking plate having at least one aperture for receiving said at least one threaded arm, and
a second nut for threaded engagement with said at least one threaded arm of said clamping member whereby said locking plate may be advanced down said clamping member to retain a ball end of a cross-link in said recess.

2. The bone anchoring member of claim 1 wherein said head further comprises
an annular enlargement having a diameter greater than the diameter of said first shaft, and a cup, said cup comprising
a body having a hole extending from a top surface to a bottom surface, said hole having a minimum diameter greater than the diameter of said first shaft and less than the diameter of said annular enlargement such that said cup is captured at said second end of said first shaft when said first end is inserted through said hole; and
wherein said second shaft extends from said top surface of said cup.

3. The bone anchoring member of claim 1 wherein said head further comprises a lateral bore and wherein said clamping member further comprises a pin, said clamping member engaging said head by insertion of said pin in said lateral bore.

4. The bone anchoring member of claim 3 wherein said clamping member is inclined from the vertical plane from 20° and 35°.

5. The bone anchoring member of claim 1 wherein said head further comprises a first lip and wherein said clamping member further comprises a second lip, said clamping member engaging said head by engagement of said first lip in said second lip.

6. The bone anchoring member of claim 1 wherein
said clamping member comprises a U-shaped clamp having a proximal base joining a first threaded arm and a second threaded arm, said first threaded arm extending through a first of said at least one through-holes of said cap and said second threaded arm extending through a second of said at least one though-holes of said cap, and
wherein said proximal base is engaged to said head.

7. The bone anchoring member of claim 6 further comprising a third nut for threaded engagement with said second threaded arm of said clamping member.

8. The bone anchoring member of claim 6 wherein said first threaded arm and said second threaded arm of said U-shaped clamp are substantially parallel and have cooperatively threaded opposing surfaces; and
wherein said second nut is externally threaded for engagement between said cooperatively threaded opposing surfaces of said first threaded arm and said second threaded arm to advance said locking plate down said clamping member in order to retain a ball end of a cross-link in said recess.

9. The anchoring member of claim 8 wherein said locking plate further defines at least one internal void for rotatably containing said second nut.

10. The anchoring member of claim 9 wherein said first threaded arm and a second threaded arm of said U-shaped clamp pass through said internal void and wherein said second nut engages said cooperatively threaded opposing surfaces within said internal void.
